(19) United States
(12) Patent Application Publication
(10) Pub. No.: US 2022/0305065 A1
(43) Pub. Date: Sep. 29, 2022

(54) HEALTH-PROMOTING BEVERAGES

(71) Applicant: Baban, Nasrollahi, Stoddard Holdings LLC, Greenwood, SC (US)

(72) Inventor: Babak Baban, Augusta, GA (US)

(21) Appl. No.: 17/704,346

(22) Filed: Mar. 25, 2022

Related U.S. Application Data
(60) Provisional application No. 63/166,034, filed on Mar. 25, 2021.

(51) Int. Cl. A61K 35/747; A23L 33/105; A23L 33/155; A23L 33/16; A23L 33/135; A23L 2/52; A61K 36/61; A61K 36/9068; A61K 36/534; A61K 36/185; A61K 31/07; A61K 31/51; A61K 31/525; A61K 31/455; A61K 31/197; A61K 31/4415; A61K 31/4188; A61K 31/519; A61K 31/714; A61K 31/375; A61K 31/593; A61K 31/355; A61K 9/00; A61K 33/34; A61K 33/26; A61K 33/06; A61K 33/24; A61K 33/32; A61K 33/00; A61K 33/04; A61K 33/30; A61K 31/716

(52) U.S. Cl. CPC A61K 35/747; A23L 33/105; A23L 33/155; A23L 33/16; A23L 33/135; A23L 2/52; A61K 36/61; A61K 36/9068; A61K 36/534; A61K 36/185; A61K 31/07; A61K 31/51; A61K 31/525; A61K 31/455; A61K 31/197; A61K 31/4415; A61K 31/4188; A61K 31/519; A61K 31/714; A61K 31/375; A61K 31/593; A61K 31/355; A61K 9/0095; A61K 33/34; A61K 33/26; A61K 33/06; A61K 33/24; A61K 33/32; A61K 33/00; A61K 33/04; A61K 33/30; A61K 31/716; A23Y 2220/73; A23Y 2220/17; A23V 2002/00

(57) ABSTRACT

Disclosed herein are health-promoting beverages. The beverages include a plurality of components that when combined a consumed provide numerous health benefits such as, for example, treating or preventing one or more symptoms caused by a bacterial or viral infection as well as enhance immune response.

### CROSS-REFERENCE TO RELATED APPLICATIONS

This application claims the benefit of and priority to co-pending U.S. Provisional Patent Application No. 63/166,034, filed on Mar. 25, 2021, the contents of which are incorporated by reference herein in their entireties.

### SUMMARY

Disclosed herein are health-promoting beverages. The beverages include a plurality of components that when combined a consumed provide numerous health benefits such as, for example, treating or preventing one or more symptoms caused by a bacterial or viral infection as well as enhance immune response.

The advantages of the invention will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein. It must be noted that, as used in the specification and the appended claims, the singular forms “a,” “an,” and “the” include plural referents unless the context clearly dictates otherwise. Thus, for example, reference to a “probiotic” includes mixtures of two or more such prebiotics, and the like.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a vitamin,” includes, but is not limited to, combinations of two or more such vitamins, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of an oxidant refers to an amount that is sufficient to achieve the desired improvement in the property modulated by the formulation component, e.g. achieving the desired level of hydrocarbon functionalization. The specific level in terms of wt % in a composition required as an effective amount will depend upon a variety of factors including the amount and type of hydrocarbon to be functionalized, amount and type of iodine-based compound, if present, amount and type of compound having the formula AₐXₙ, if present, and temperature of the reaction vessel.

As used herein, the terms “optional” or “optionally” means that the subsequently described event or circumstance can or cannot occur, and that the description includes instances where said event or circumstance occurs and instances where it does not.

As used herein the terms “weight percent,” “wt %,” and “wt. %,” which can be used interchangeably, indicate the percent by weight of a given component based on the total weight of the composition, unless otherwise specified. That is, unless otherwise specified, all wt % values are based on the total weight of the composition. It should be understood that the sum of wt % values for all components in a disclosed composition or formulation are equal to 100.

References in the specification and concluding claims to parts by weight of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc., of these materials are disclosed, that while specific reference to each individual and collective combination and permutation of these compounds may not be explicitly disclosed, each is specifically contemplated and described herein. For example, if a vitamin is disclosed and discussed and a number of different minerals are discussed, each and every combination of vitamin and mineral that is possible is specifically contemplated unless specifically indicated to the contrary. For example, if a class of vitamins A, B, and C are disclosed, as well as a class of minerals D, E, and F, and an example combination of A+D is disclosed, then even if each is not individually recited, each is individually and collectively contemplated. Thus, in this example, each of the combinations A+E, A+F, B+D, B+E, B+F, C+D, C+E, and C+F is specifically contemplated and should be considered from disclosure of A, B, and C; D, E, and F; and the example combination of A+D. Likewise, any subset or combination of these is also specifically contemplated and disclosed. Thus, for example, the sub-group of A+E, B+F, and C+E is specifically contemplated and should be considered from disclosure of A, B, and C; D, E, and F; and the example combination of A+D. This concept applies to all aspects of the disclosure including, but not limited to, steps in methods of making and using the disclosed compositions. Thus, if there are a variety of additional steps that can be performed with any specific embodiment or combination of embodiments of the disclosed methods, each such combination is specifically contemplated and should be considered disclosed.

The term “treat” as used herein is defined as maintaining or reducing the symptoms of a pre-existing condition (e.g., infection) in a subject when compared to the same pre-existing condition when the beverage described herein is not consumed by the subject.

The term “prevent” as used herein is defined as eliminating or reducing the likelihood of the occurrence of one or more symptoms of a disease or disorder in a subject when compared to the same pre-existing condition when the beverage described herein is not consumed by the subject.

The term “enhance” as used herein is defined as increasing a response (e.g., immune) in a subject when compared to the same response when the beverage described herein is consumed by the subject.

A “pharmaceutically-acceptable compound” is used to refer to a neutral complex. In some aspects, a pharmaceutically-acceptable compound may be more economical to produce, may have increased chemical stability, may allow manipulation of the compound's pharmacokinetics and bioavailability, may make the compound easier to administer, or a combination thereof. In a further aspect, a pharmaceutically-acceptable compound can alter a compound's dissolution or solubility. In one aspect, the pharmaceutically-acceptable compound can be an ionic compound (or “pharmaceutically-acceptable salt”). For example, a pharmaceutically acceptable compound can be a reaction product between an organic acid (e.g., citric acid) and base (e.g., zinc hydroxide) to produce the ionic compound zinc citrate.

Described herein are health-promoting beverages. Each component used to produce the beverages described herein and methods for producing and using the same are described below.

### Eucalyptus

The beverages disclosed herein include eucalyptus. Eucalyptus can help break up congestion and to help to expel phlegm from the respiratory system. It has been used in traditional medicine worldwide as anti-inflammatory mixtures for the symptoms of respiratory infections, such as sinus congestion, flu and common cold. Eucalyptus byproducts have been applied to cure pharyngitis, bronchitis, and sinusitis. In one aspect, eucalyptus is present in the amount of from about 0.5 mg to about 20 mg per 8 ounce serving of the beverage, or about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg, where any value can be a lower or upper endpoint of a range (e.g., about 1 mg to about 8 mg, about 4 mg to about 6 mg, etc.).

### Ginger or Ginger Extract

The beverages disclosed herein include ginger or ginger extract. Ginger has antiviral activity due to its high concentration of potent plant compounds. Additionally, specific compounds in ginger, such as gingerols and zingerone, can inhibit viral replication and prevent viruses from entering host cells. Further, ginger improves circulation to the lungs, reduces inflammation, and helps to break down mucus. In one aspect, ginger is present in the amount of from about 0.1 g to about 2 g per 8 ounce serving of the beverage, or about 0.1 g, about 0.2 g, about 0.3 g, about 0.4 g, about 0.5 g, about 0.6 g, about 0.7 g, about 0.8 g, about 0.9 g, about 1.0 g, about 1.1 g, about 1.2 g, about 1.3 g, about 1.4 g, about 1.5 g, about 1.6 g, about 1.7 g, about 1.8 g, about 1.9 g, or about 2.0 g, where any value can be a lower or upper endpoint of a range (e.g., about 0.1 g to about 1.0 g, about 0.4 g to about 0.6 g, etc.).

### Peppermint

The beverages disclosed herein include peppermint. Peppermint has antibacterial, antiviral and anti-inflammatory properties, which can prevent clogged sinuses due to infections, the common cold and allergies. Peppermint also contains menthol, which is known to help to relax the muscles of the respiratory system and facilitate breathing. In one aspect, peppermint is present in the amount of from about 5 mg to about 100 mg per 8 ounce serving of the beverage, or about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, where any value can be a lower or upper endpoint of a range (e.g., about 25 mg to about 75 mg, about 40 mg to about 60 mg, etc.).

### Elderberry

The beverages disclosed herein include Elderberry. Elderberry possess several unique health based on plant nutrients such as pigment flavonoid antioxidants, anthocyanins, minerals, and vitamins that contribute immensely towards robust health and wellness. Elderberry can also reduce cold duration and symptoms. In one aspect, elderberry is present in the amount of from about 1 g to about 20 g per 8 ounce serving of the beverage, or about 1 g, about 2 g, about 3 g, about 4 g, about 5 g, about 6 g, about 7 g, about 8 g, about 9 g, about 11 g, about 12 g, about 13 g, about 14 g, about 15 g, about 16 g, about 17 g, about 18 g, about 19 g, or about 20 g, where any value can be a lower or upper endpoint of a range (e.g., about 5 g to about 15 g, about 8 g to about 12 g, etc.). Ire another aspect, elderberry is present in the amount of from about 1 mg to about 20 mg per 8 ounce serving of the beverage, or about 1 mg, about 2 mg, about 3 mg, about 4 mg, about 5 mg, about 6 mg, about 7 mg, about 8 mg, about 9 mg, about 11 mg, about 12 mg, about 13 mg, about 14 mg, about 15 mg, about 16 mg, about 17 mg, about 18 mg, about 19 mg, or about 20 mg, where any value can be a lower or upper endpoint of a range (e.g., about 5 mg to about 15 mg, about 8 mg to about 12 mg, etc.).

### Vitamins

As used herein, a vitamin is an organic compound that is required in small quantities to support essential body functions including growth and cellular maintenance. In some aspects, vitamins cannot be produced by the body (e.g., vitamin C) or are not produced in sufficient quantities to meet a patient's needs and must be supplied via external sources. In one aspect, many diets are insufficient in one or more key vitamins and supplements are often indicated. In one aspect, a vitamin can act as a cofactor to assist an enzyme in carrying out an essential biochemical reaction. In one aspect, the beverages disclosed herein include one or more vitamins. In a further aspect, the vitamin can be vitamin A, B₁, B2, B₃, B₅, B₆, B₇, B₉, B₁₂, C, D₃, E, or K. Ina still further aspect, an 8 ounce serving of the beverage contains 100% of the recommended daily intake of any vitamins contained therein. In one aspect, the vitamin is a combination of vitamin B6 and D. In another aspect, the beverage does not include vitamin C.

### Vitamin A

In one aspect, the beverages disclosed herein include vitamin A. Vitamin A can be important for maintenance of the immune system and for low-light and color vision. Vitamin A can also act as an antioxidant. In one aspect, the vitamin A source is retinyl palmitate, which is converted to retinol by the body, or is beta-carotene, or a combination thereof. In one aspect, the amount of retinyl palmitate or other retinol source per 8 ounce serving of the beverages provides from about 500 to about 1500 μg of vitamin A, or about 500 μg, about 600 μg, about 700 μg, about 800 μg, about 900 μg, about 1,000 μg, about 1,100 μg, about 1,200 μg, about 1,300 μg, about 1,400 μg, or about 1,500 μg, where any value can be a lower or upper endpoint of a range (e.g., about 700 μg to about 1,200 μg, etc.). In another aspect, the amount of beta carotene per 8 ounce serving of the beverages can be higher than about 1500 μg because the subject's body converts only the amount needed to retinol. In still another aspect, a mixture of the retinol and beta-carotene forms of vitamin A is included in the beverages disclosed herein.

### B Complex Vitamins

In one aspect, the beverages disclosed herein include one or more B vitamins. B vitamins generally act as cofactors or coenzymes or precursors needed to make cofactors or coenzymes. B vitamins are not stored in the body and must be regularly supplied by dietary or other means to avoid deficiency.

Low levels of B vitamins can affect the manufacture of neurotransmitters and contribute to stress and anxiety. Here, supplementation of B vitamins can reduce stress related to their deficiency. Low levels of B vitamins can be caused by kidney dialysis, the use of diuretics, and overconsumption of refined carbohydrates, since digestion of many carbohydrates requires the use of B vitamins. Inadequate B vitamin intake is linked to blood sugar surges in patients who consume too many refined carbohydrates.

In one aspect, thiamin or vitamin B₁ is included in the beverages disclosed herein. Thiamin may be important to nerve and muscle health, production of hydrochloric acid in the stomach, and treatment of constipation and fatigue, as well as assisting digestion of some carbohydrates and proteins. In one aspect, the thiamine source is thiamine mononitrate. In one aspect, the amount of vitamin B₁ present per 8 ounce serving of the beverages is from about 0.1 mg to about 50 mg, or about 0.1 mg, about 5 mg, about 10 mg, about 15 mg, about 20 mg, about 25 mg, about 30 mg, about 35 mg, about 40 mg, about 45 mg, or about 50 mg, where any value can be a lower or upper endpoint of a range (e.g., about 10 mg to about 40 mg, about 15 mg to about 30 mg, etc.).

In another aspect, riboflavin, or vitamin B₂, is included in the beverages disclosed herein. Riboflavin may be important for growth, red blood cell production, and eye health, as well as assisting in the digestion of some carbohydrates, fats, ketone bodies, and proteins. In one aspect, the riboflavin source is molecular riboflavin. In one aspect, the amount of vitamin B2 present per 8 ounce serving is from about 0.1 mg to about 2 mg, or about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg, where any value can be a lower or upper endpoint of a range (e.g., about 0.5 mg to about 1.5 mg, about 1.0 mg to about 1.8 mg, etc.).

In still another aspect, nicotinic acid or nicotinamide, also known as niacinamide or vitamin B₃, is included in the beverages disclosed herein. Vitamin B₃ is important to digestive system health and may assist in the digestion of some carbohydrates, as well as in the production of various sex and stress-related hormones. Niacin may be useful in reducing cholesterol levels in the blood. In one aspect, the niacinamide source is molecular niacinamide. In a further aspect, the amount of vitamin B3 present per 8 ounce serving is from about 1 mg to about 40 mg, or about 1 mg, about 5 mg, about 10 mg, about 15 mg, about 20 mg, about 25 mg, about 30 mg, about 35 mg, or about 40 mg, where any value can be a lower or upper endpoint of a range (e.g., about 1 mg to about 30 mg, about 15 mg to about 35 mg, etc.).

In one aspect, pantothenic acid, also known as vitamin B₅, is included in the beverages disclosed herein. Vitamin B₅ is important in the metabolism of macronutrients such as proteins, fats, and carbohydrates, and is required for the synthesis of coenzyme A. Pantothenic acid is also important for acylation and acetylation in signal transduction and other biological functions including enzyme activation. In one aspect, the pantothenic acid source is pantothenol or calcium pantothenate. In a further aspect, the amount of vitamin B₅ present per 8 ounce serving is from about 1 mg to about 10 mg, or about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg, where any value can be a lower or upper endpoint of a range (e.g., about 2 mg to about 9 mg, about 3.5 mg to about 7.5 mg, etc.).

In still another aspect, the beverages disclosed herein include pyridoxal phosphate (also occurring as pyridoxine), or vitamin B₆. Vitamin B₆ deficiency can result in a weakened immune system and a decrease in the production of serum antibodies, which can make the host more susceptible to the viral and respiratory infections. In one aspect, the pyridoxine source is pyridoxine hydrochloride. In one aspect, the amount of vitamin B6 present per 8 ounce serving is from about 0.1 mg to about 2 mg, or about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, or about 2.0 mg, where any value can be a lower or upper endpoint of a range (e.g., about 0.5 mg to about 1.5 mg, about 1.0 mg to about 1.8 mg, etc.).

In still another aspect, the beverages disclosed herein include biotin, or vitamin B₇. Biotin is important to various aspects of metabolism and may be important in strengthening the hair and nails as well as in the metabolism of fats and amino acids. In one aspect, the biotin source is molecular biotin. In one aspect, the amount of vitamin B₇ present per 8 ounce serving is from about 10 μg to about 500 μg, or about 10 μg, about 50 μg, about 100 μg, about 150 μg, about 200 μg, about 250 μg, about 300 μg, about 350 μg, about 400 μg, about 450 μg, or about 500 μg, where any value can be a lower or upper endpoint of a range (e.g., about 50 μg to about 400 μg, etc.).

In one aspect, the beverages disclosed herein include folate, also known as folic acid or vitamin B₉. Folic acid may be important for brain function, mental health, red blood cell production, and production of nucleic acids. In one aspect, the folate source is folic acid. In a further aspect, the amount of vitamin B₉ present per 8 ounce serving of the supplement is from about 0.05 mg to about 1 mg, or about 0.05 mg, about 0.1 mg, about 0.2 mg, about 0.3 mg, about 0.4 mg, about 0.5 mg, about 0.6 mg, about 0.7 mg, about 0.8 mg, about 0.9 mg, or about 1.0 mg, where any value can be a lower or upper endpoint of a range (e.g., about 0.05 mg to about 0.8 mg, about 0.2 mg to about 0.6 mg, etc.).

In another aspect, cobalamin, or vitamin B₁₂, is included in the beverages disclosed herein. Cobalamin may be present as the cyanocobalamin, hydroxycobalamin, adenosylcobalamin, or methylcobalamin form. Cobalamin is important to nervous system health, the production of red blood cells, and synthesis of nucleic acids. In a further aspect, cobalamin works synergistically with folate with respect to red blood cell production. In one aspect, the cobalamin source is cyanocobalamin. In a further aspect, the amount of vitamin B₁₂ present per 8 ounce serving of the beverage is from about 1 μg to about 150 μg, or about 1 μg, about 5 μg, about 10 μg, about 15 μg, about 20 μg, about 25 μg, about 30 μg, about 35 μg, about 40 μg, about 45 μg, about 50 μg, about 60 μg, about 70 μg, about 80 μg, about 90 μg, about 100 μg, about 110 μg, about 120 μg, about 130 μg, about 140 μg, or about 150 μg (e.g., about 10 μg to about 100 μg, etc.).

### Vitamin D

In one aspect, the beverages disclosed herein include vitamin D. Vitamin D is a fat-soluble vitamin that is usually activated by healthy or well-functioning kidneys. Vitamin D may improve bone health and immune system function and may protect against certain cancers. Vitamin D can increase calcium and phosphate absorption from the small intestine, is important in bone mineralization, and maintains proper calcium and phosphorus levels in the serum. In one aspect, the beverages disclosed herein include a source of vitamin D. In a further aspect, the source of vitamin D is vitamin D₃, or cholecalciferol.

In one aspect, the amount of vitamin D present per 8 ounce serving of the beverage is from about 1 μg to about 20 μg, or about 1 μg, about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, or about 20 μg (e.g., about 1 μg to about 10 μg, etc.).

### Vitamin E

In one aspect, the beverages disclosed herein include vitamin E. Vitamin E is a fat-soluble vitamin found naturally in plant oils such as corn oil, soybean oil, wheat germ oil, sunflower oil, and safflower oil. Vitamin E can act as an antioxidant and can neutralize reactive oxygen species that would otherwise cause cell or tissue damage. Vitamin E deficiency, although rare, can cause various disorders of neural and nervous tissues including macular degeneration, retinopathy, and poor conduction of electrical impulses along nerves. Vitamin E may also protect lipids, including cell membrane lipids, from oxidation. Finally, vitamin E may have a role in reducing inflammation. In one aspect, the vitamin E included herein can be in the form of a tocopherol (α, β, γ, or δ), a tocotrienol (α, β, γ, or δ), or a combination of any of these. In one aspect, the vitamin E is extracted from a plant source.

In one aspect, the amount of vitamin E present per 8 ounce serving of the beverage is from about 5 to about 30 mg, or about 5 mg, about 10 mg, about 15 mg, about 20 mg, about 25 mg, or about 30 mg, where any value can be a lower or upper endpoint of a range (e.g., about 5 mg to about 25 mg, etc.).

### Vitamin K

In one aspect, the beverages disclosed herein include vitamin K. Vitamin K is a fat-soluble vitamin required for synthesis of proteins involved in blood coagulation and/or for controlling calcium binding in tissues. In one aspect, the beverages include vitamin K₁ or phylloquinone, which is converted to vitamin K₂ by gut bacteria. In an alternative aspect, the beverages include vitamin K₂ or menaquinone. In some aspects, vitamin K supplementation is contraindicated (such as for patients taking warfarin) and so, in some aspects, the beverages do not include a source of vitamin K.

In one aspect, when included, the amount of vitamin K present per 8 ounce serving of the beverage is from about 50 μg to about 150 μg of vitamin K, or about 1 μg, about 5 μg, about 10 μg, about 15 μg, about 20 μg, about 25 μg, about 30 μg, about 35 μg, about 40 μg, about 45 μg, about 50 μg, about 60 μg, about 70 μg, about 80 μg, about 90 μg, about 100 μg, about 110 μg, about 120 μg, about 130 μg, about 140 μg, or about 150 μg (e.g., about 10 μg to about 100 μg, etc.), where any value can be a lower or upper endpoint of a range (e.g., about 10 μg to about 100 μg, etc.).

### Minerals

As used herein, a mineral is an inorganic element that is obtained from food or supplementation and is required for the functioning of the human body. Minerals include, but are not limited to, calcium, chromium, copper, iron, magnesium, manganese, molybdenum, nickel, potassium, selenium, sodium, vanadium, cobalt, and zinc. In one aspect, a mineral can act as a cofactor. In other aspects, minerals can be used for cell signaling, or can be essential structural components of the body (e.g., calcium in bone), or can associate with proteins, nucleic acids, lipids, and carbohydrates to maintain particular secondary, tertiary, and quaternary structures.

In one aspect, the mineral is a pharmaceutically-acceptable salt. In another aspect, the mineral can be part of a chelate complex. Chelates have a cyclic structure in which a central metallic ion is held tight via covalent-coordinate bonds to form a coordinate compound or chelate complex. Furthermore, chelates occur abundantly in nature; for example, chlorophyll is a chelate of magnesium, hemoglobin of iron, vitamin B₁₂ with cobalt, hemocyanin with copper, and enzymes that contain vanadium or molybdenum.

### Calcium

In one aspect, the beverages disclosed herein include a pharmaceutically-acceptable compound of calcium. Calcium supplementation can be useful in preventing osteoporosis since calcium is a major component of bones and teeth. Calcium's absorption and effects are enhanced or aided by vitamin D and parathyroid hormone. Calcium may be important to B complex vitamin absorption and is important to the functional integrity of many cells. Moreover, calcium requirements may increase with increased protein consumption.

In one aspect, the calcium is provided as an ionic compound of calcium. Examples of such compounds include, but are not limited to, calcium citrate, calcium citrate tetrahydrate, calcium lactate pentahydrate, and calcium ascorbate. In one aspect, the beverages disclosed herein include an ionic compound of calcium in an amount such that there are about 100 mg to about 2,500 mg, or about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, or about 2500 mg, where any value can be a lower or upper endpoint of a range (e.g., about 200 mg to about 1800 mg, about 500 mg to about 1500 mg, etc.). For example, calcium citrate tetrahydrate has a molecular weight of 570.49, of which calcium is 21.08%. Thus, in order for the beverage to have 500 mg of calcium per serving, there will be approximately 2,372 mg of calcium citrate tetrahydrate per serving. In another aspect, 250 to 500 mg of calcium is provided per 8 ounce serving of the beverages disclosed herein.

### Chromium

In one aspect, the beverages disclosed herein include a pharmaceutically-acceptable compound of chromium. Chromium is required in trace amounts in the human diet. In a still further aspect, trivalent (3+) chromium is the biologically active and non-toxic oxidation state of chromium. Chromium can enhance the action of insulin and may be involved in the metabolism of macronutrients including carbohydrates, protein, and fats.

In one aspect, the chromium is provided as an ionic compound of chromium or as an extract from a natural source of chromium. Further in this aspect, the chromium can be provided as chromium chloride, chromium nicotinate, chromium picolinate, high-chromium yeast, or chromium citrate. In one aspect, the chromium is in the form of chromium picolinate. In one aspect, the beverages disclosed herein include from about 1 to about 10 μg of chromium, or about 1 μg, about 2 μg, about 3 μg, about 4 μg, about 5 μg, about 6 μg, about 7 μg, about 8 μg, about 9 μg, or about 10 μg, where any value can be a lower or upper endpoint of a range (e.g., about 3 μg to about 8 μg, etc.)

### Cobalt

In another aspect, the beverages disclosed herein include a pharmaceutically-acceptable compound of cobalt. Cobalt is a central component of cobalamin or vitamin B₁₂. The primary biological function of cobalt relates to the function of vitamin B₁₂ as discussed above.

In one aspect, the cobalt is provided as part of a cobalamin compound, wherein the cobalt is complexed by four nitrogen atoms and an additional substituent which may be a cyano group (as in cyanocobalamin), a hydroxyl group (as in hydroxocobalamin), a methyl group (as in methylcobalamin), or an adenosyl group (as in adenosylcobalamin). In one aspect, the cobalt source is adenosylcobalamin or methylcobalamin. In a further aspect, the amount of cobalt included in the beverages disclosed herein is dictated by the amount of vitamin B₁₂ included as outlined previously.

### Copper

In one aspect, the formulations disclosed herein include a pharmaceutically-acceptable compound of copper. Biological copper is important in electron transport and oxygen transport and is found in cytochrome c oxidase in mitochondria. Copper is a component of superoxide dismutases and may aid in iron absorption.

In one aspect, the copper is provided as an ionic compound of copper. Further in this aspect, the copper can be provided as copper oxide, copper chloride, copper gluconate, copper sulfate, or a copper amino acid chelate. In one aspect, the chromium is in the form of cupric oxide. In one aspect, the beverages disclosed herein include from about 100 μg to about 200 μg of copper, or about 100 μg, about 110 μg, about 120 μg, about 130 μg, about 140 μg, about 150 μg, about 160 μg, about 170 μg, about 180 μg, about 190 μg, or about 200 μg, where any value can be a lower or upper endpoint of a range (e.g., about 10 μg to about 100 μg, etc.).

### Iron

In one aspect, the formulations disclosed herein include a pharmaceutically-acceptable compound of iron. Iron is vital in preventing anemia. Iron is biochemically important to oxygen and electron transport and may improve the quality and quantity of red blood cells. Heme iron is found in meats and is well-absorbed, while non-heme iron is poorly absorbed. Supplementation of iron may be particularly important for patients consuming diets that include little or no meat.

In one aspect, the iron is provided as an ionic compound of iron such as, for example, iron gluconate, iron gluconate dehydrate, or iron sulfate heptahydrate. In one aspect, the beverages disclosed herein include an ionic compound of iron in an amount such that there are about 1 mg to about 45 mg, or about 1 mg, about 5 mg, about 10 mg, about 15 mg, about 20 mg, about 25 mg, about 30 mg, about 35 mg, about 40 mg, or about 45 mg, where any value can be a lower or upper endpoint of a range (e.g., about 10 mg to about 30 mg, etc.).

### Magnesium

In one aspect, the beverages disclosed herein include a pharmaceutically-acceptable compound of magnesium. Magnesium is necessary for the proper functioning of calcium in the body such as, for example, assisting in entry of calcium ions into cells, thus preventing calcification of tissues. Magnesium supplementation may support healthy heart function.

Magnesium is required for normal bone structure formation and the functioning of several hundred enzymes, especially those with ATP-dependent phosphorylation, protein synthesis, and carbohydrate metabolism functions. Magnesium in the extracellular matrix is important to electrical potentials in nerve and muscle cells and the transmission of impulses across neuromuscular junctions.

In one aspect, the magnesium is provided as an ionic compound of magnesium. Examples of such compounds include, but are not limited to, magnesium citrate, magnesium sulfate monohydrate or heptahydrate, magnesium acetate tetrahydrate, magnesium D-gluconate hydrate, or magnesium nitrate hexahydrate. In one aspect, the beverages described herein include an ionic compound of magnesium in an amount such that there are about 100 to about 500 mg, or about 100 mg, about 150 mg, about 200 mg, about 250 mg, about 300 mg, about 350 mg, about 400 mg, about 450 mg, or about 500 mg, where any value can be a lower or upper endpoint of a range (e.g., about 200 mg to about 400 mg, etc.).

### Manganese

In one aspect, the beverages disclosed herein include a pharmaceutically-acceptable compound of manganese. Manganese is a required cofactor for various oxidoreductases, transferases, hydrolases, lyases, isomerases, ligases, lectins, and integrins, as well as a magnesium-containing superoxide dismutase. Manganese is also an important mineral for bone health including in cartilage formation and bone mineralization.

In another aspect, the manganese is provided as an ionic compound of manganese. Examples of such compounds include, but are not limited to, manganese amino acid chelates, manganese ascorbate, manganese chloride, manganese chloride tetrahydrate, manganese citrate, manganese dioxide, manganese gluconate, manganese glycerophosphate, manganese sulfate, manganese sulfate monohydrate, and manganese sulfate tetrahydrate. In one aspect, the beverages disclosed herein include an ionic compound of manganese such that there are about 1 mg to about 5 mg, or about 1 mg, about 1.5 mg, about 2 mg, about 2.5 mg, about 3 mg, about 3.5 mg, about 4 mg, about 4.5 mg, or about 5 mg, where any value can be a lower or upper endpoint of a range (e.g., about 1.5 mg to about 4.5 mg, etc.).

### Molybdenum

In one aspect, the beverages disclosed herein include a pharmaceutically-acceptable salt of molybdenum. Molybdenum is a heteroatom at the active site of certain enzymes such as, for example, sulfite oxidase, xanthine oxidoreductase, aldehyde oxidase, and mitochondrial amidoxime reductase, and molybdenum is used by the body in catabolism of proteins. Lack of molybdenum in the diet has been associated with higher rates of esophageal cancer.

In one aspect, the molybdenum is provided as an ionic compound of molybdenum or molybdate including, but not limited to, ammonium molybdate, sodium molybdate, molybdenum citrate, or molybdenum picolinate. In one aspect, the beverages disclosed herein include an ionic compound of molybdenum or molybdate such that there are about 10 μg to about 100 μg, or about 100 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, or about 100 μg, where any value can be a lower or upper endpoint of a range (e.g., about 20 μg to about 80 μg, etc.).

### Nickel

In one aspect, the beverages disclosed herein include a pharmaceutically-acceptable salt of nickel. Nickel acts as an essential nutrient for bacteria residing in the large intestine, and is an important cofactor for several bacterial enzymes. Nickel may be important for increasing iron absorption and preventing anemia, and may also have a role in strengthening osteoporotic bones.

In one aspect, the nickel is provided as an ionic compound of nickel including, but not limited to, nickel chloride or nickel sulfate. In a further aspect, the beverages disclosed herein include an ionic compound of nickel such that there are from about 0.1 to about 1 mg of nickel, or about 0.1 mg, about 0.2 mg, about 0.3 mg, about 0.4 mg, about 0.5 mg, about 0.6 mg, about 0.7 mg, about 0.8 mg, about 0.9 mg, or about 1.0 mg, where any value can be a lower or upper endpoint of a range (e.g., about 0.2 mg to about 0.8 mg, etc.).

### Potassium

In one aspect, the formulations disclosed herein include a pharmaceutically-acceptable salt of potassium. A diet low in potassium can put one at risk for hypertension and/or hypokalemia. In a still further aspect, potassium supplementation can decrease blood pressure in people with hypertension and can reduce the risk of stroke.

In another aspect, the potassium is provided as an ionic compound of potassium including, but not limited to, potassium acetate, potassium bicarbonate, potassium chloride, potassium citrate, potassium gluconate, potassium glycerophosphate, potassium orotate, potassium phosphate, potassium ascorbate, potassium hydroxide, or potassium sulfate. In a still further aspect, the beverages disclosed herein include an ionic compound of potassium such that there are from about 2.5 to about 7.5 mg of potassium, or about 2.5 mg, about 3 mg, about 3.5 mg, about 4 mg, about 4.5 mg, about 5 mg, about 5.5 mg, about 6 mg, about 6.5 mg, about 7 mg, or about 7.5 mg, where any value can be a lower or upper endpoint of a range (e.g., about 3 mg to about 7 mg, etc.).

### Selenium

In one aspect, the beverages disclosed herein include a pharmaceutically-acceptable compound of selenium. Selenium is a powerful antioxidant and is especially useful when employed against the damaging effects of free radicals. Selenium supplementation may protect against hardening of the arteries and harmful molecules.

In one aspect, the selenium is provided as an ionic compound of selenium such as, for example, sodium selenate. In a further aspect, the formulations described herein include an ionic compound of selenium in an amount such that there are about 10 μg to about 400 μg, or about 10 μg, about 50 μg, about 100 μg, about 150 μg, about 200 μg, about 250 μg, about 300 μg, about 350 μg, or about 400 μg, where any value can be a lower or upper endpoint of a range (e.g., about 100 μg to about 300 μg, etc.).

### Sodium

Sodium is an essential mineral that regulates blood pressure and blood volume. However, sodium is a major contributor to hypertension and is found in excess in many Western diets. In one aspect, the beverages disclosed herein do not include sodium. In another aspect, the beverages disclosed herein include no more than 500 mg of sodium, or include less than 50 mg of sodium, or include essentially 0 mg of sodium per 8 ounce serving of the beverages.

### Vanadium

In one aspect, the beverages disclosed herein include a pharmaceutically-acceptable compound of vanadium. Vanadium can be used for treating diabetes and prediabetes, edema or water retention, heart disease, and high cholesterol. Vanadium can act like insulin or helps enhance the effects of insulin.

In another aspect, the beverages disclosed herein are supplemented with a pharmaceutically-acceptable salt of vanadium such as, for example, vanadium pentoxide, vanadyl sulfate, or vanadyl nicotinate. In a further aspect, the beverages disclosed herein include an ionic compound of vanadium containing from about 0.25 to about 1.5 mg of vanadium, or about 0.25 mg, about 0.5 mg, about 0.75 mg, about 1 mg, about 1.25 mg, or about 1.5 mg, where any value can be a lower or upper endpoint of a range (e.g., about 0.5 mg to about 1 mg, etc.).

### Zinc

In one aspect, the beverages disclosed herein include a pharmaceutically-acceptable compound of zinc. Zinc can have anti-inflammatory and anti-depressant effects or may support the immune system. Zinc is crucial for immune regulation in human body. The immune system needs zinc to activate T lymphocytes (T cells), which are a major component in cellular (adoptive/acquired) immunity. Adoptive immunity is a major part of immune system against any viral infection and intra-cellular diseases (e.g., cancer), where any deficiency in zinc impairs the immune system.

Examples of pharmaceutically-acceptable compounds of zinc include, but are not limited to, zinc citrate, zinc citrate dehydrate, zinc acetate dehydrate, or zinc nitrate hexahydrate. In one aspect, the beverages disclosed herein include an ionic compound of zinc in an amount such that there is about 1 mg to about 20 mg, or about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg, where any value can be a lower or upper endpoint of a range (e.g., about 3 mg to about 15 mg, about 4 mg to about 6 mg etc.).

### Probiotics

The beverages disclosed herein include a probiotic. In one aspect, the probiotic is a live microorganism that can colonize in the digestive system. In another aspect, the probiotic is a microbial spore that is shelf stable in aqueous solution and can survive the harsh environment of the upper gastrointestinal tract in order to establish a population in the gut.

In one aspect, the probiotic can be Bacillus coagulans, Lactobacillus plantarum 299v (Lp299v), or a combination thereof. In a further aspect, when the probiotic is B. coagulans, it is the strain identified as GBI-30, 6086 in the NCBI BioSample database.

In one aspect, the beverages disclosed herein include additional probiotics. In one aspect, the additional probiotic is a yeast such as, for example, an organism selected from the genera Saccharomyces, Debaromyces, Candida, Pichia, or Torulopsis. In an alternative aspect, the probiotic is a mold such as, for example, an organism selected from the genera Aspergillus, Rhizopus, Mucor, or Penicillium. In still another aspect, the probiotic is a bacterium such as, for example, an organism selected from the genera Bifidobacterium, Bacterioides, Clostridium, Fusobacterium, Melissococcus, Propionibacterium, Streptococcus, Enterococcus, Lactococcus, Kocuria, Staphylococcus, Peptostreptococcus, Bacillus, Pediococcus, Micrococcus, Euconostoc, Weissella, Aerococcus, Oenococcus, or Lactobacillus. In still another aspect, the additional probiotic is a combination of organisms from one or more of the genera listed above.

In another aspect, the additional probiotic is Aspergillus niger, A. oryzae, Bacillus coagulans, B. lentus, B. licheniformis, B. mesentericus, B. pumilus, B. subtilis, B. natto, Bacteroides amylophilus, Bac. capillosus, Bac. ruminocola, Bac. suis, Bifidobacterium adolescentis, Bif. animalis, Bif. breve, Bif. bifidum, Bif. infantis, Bif. lactis, Bif. longum, Bif. pseudolongum, Bif. thermophilum, Candida pintolepesii, Clostridium butyricum, Enterococcus cremoris, E. diacetylactis, E. faecium, E. intermedius, E. lactis, E. mundtii, E. thermophilus, Escherichia coli, Kluyveromyces fragilis, Lactobacillus acidophilus, L. alimentarius, L. amylovorus, L. crispatus, L. bifidus, L. brevis, L. bulgaricus, L. casei, L. curvatus, L. cellobiosus, L. delbrueckii ss. bulgaricus, L. farciminis, L. fermentum, L. gasseri, L. helveticus, L. lactis, L. plantarum, L. johnsonii, L. reuteri, L. rhamnosus, L. sakei, L. salivarius, Leuconostoc mesenteroides, Pediococcus cereviseae (damnosus), P. acidlactici, P. pentosaceus, Propionibacterium freudenreichii, Prop. shertnanii, Saccharomyces cereviseae, Staphyloccus carnosus, Staph. xylosus, Streptococcus infantarius, Strep. salivarius ss. thermophilus, Strep. thermophilus, Strep. lactis, or any combination thereof.

As used herein, a “colony forming unit” or “CFU” is a measure of how many bacteria in a sample are capable of forming colonies. In this aspect, microorganisms are cultured and only viable cells are counted as CFUs.

In one aspect, the beverages disclosed herein contain from one billion to ten billion CFU of the probiotic microorganism. In another aspect, the beverages contain about 1 billion, about 1.5 billion, about 2 billion, about 2.5 billion, about 3 billion, about 3.5 billion, about 4 billion, about 4.5 billion, about 5 billion, about 5.5 billion, about 6 billion, about 6.5 billion, about 7 billion, about 7.5 billion, about 8 billion, about 8.5 billion, about 9 billion, about 9.5 billion, or about 10 billion CFU per 8 ounces of beverage, where any value can be a lower or upper endpoint of a range (e.g., about one billion to nine billion, about five billion to eight billion, etc.)

### Additional Components

In some aspects, the beverages disclosed herein include additional ingredients known in the art. In other aspects, the beverages are 100% natural (i.e., made with 100% natural components) with no artificial colors, dyes, preservatives, sweeteners, or the like.

In one aspect, the beverages are formulated with no ingredients from genetically-modified organisms. In another aspect, the beverages are gluten free, lactose/dairy free, and vegan.

In one aspect, the additional ingredients are selected from polydextrose, milk protein concentrate, sodium citrate, malic acid, tapioca starch, corn starch, calcium caseinate, potassium carbonate, vegetable juice, salt or sea salt, carrageenan, whey protein concentrate, fruit juice concentrate, cane sugar or another sweetener, chicory root fiber, fruit pectin, locust bean gum, milk, natural flavors, preservatives, or any combination thereof. In another aspect, the additional ingredients are selected from one or more of the following categories:

### Sweetener

A “sweetener” is a compound, extract, or composition that imparts a sweet taste to a food of beverage. Inclusion of a sweetener may assist in improving the flavor of a food or beverage product. In one aspect, the beverages disclosed herein do not contain any added sweeteners. In another aspect, the beverages disclosed herein contain one or more added sweeteners.

In one aspect, the sweetener is a natural sweetener. Examples of sweeteners useful herein include, but are not limited to, cane sugar, beet sugar, coconut sugar, agave nectar, honey, maple syrup, molasses, barley malt syrup, brown rice syrup, date sugar, xylitol, erythritol, lucuma powder, monk fruit extract, stevia extract, or any combination thereof. In one aspect, the sweetener can add calories or other nutrients such as, for example, fiber to the beverages. In an alternative aspect, the sweetener is a non-caloric sweetener.

In one aspect, the sweetener or other additional ingredients adds a small number of calories to the beverages disclosed herein. In one aspect, the beverage has from 5 calories to 20 calories per 8 ounce serving, or from 10 calories to 20 calories per serving, or from 10 calories to 15 calories per serving, or has about 10 calories per serving.

### Preservative

A “preservative” is a compound or composition that is added to food and/or beverage products to prevent spoilage and prolong shelf life. In one aspect, a preservative can be an artificial or added preservative, such as, for example, sodium benzoate, benzoic acid, a nitrite, a sulfite, sodium sorbate, potassium sorbate, or a similar compound. In one aspect, added preservatives are not found in natural products. In another aspect, the beverages described herein do not contain any added or artificial preservatives.

In one aspect, the beverages described herein include a natural preservative. “Natural preservatives” as described herein include antioxidants such as, for example, vitamins A, C, and E (often present as “mixed tocopherols”). In another aspect, the natural preservative can be rosemary oil or rosemary leaf extract, lemon juice, cinnamon extract, citric acid, or a combination thereof.

### Natural Flavors

“Natural flavors” are defined by the Code of Federal Regulations as essential oils, oleoresins, essences or extractives, protein hydrolysates, distillates, or any products of roasting, heating, or enzymolysis, which contain the flavoring constituents derived from a spice, fruit or fruit juice, vegetable or vegetable juice, edible yeast, herb, bark, bud, root, leaf, or similar plant material, or meat, seafood, poultry, eggs, dairy products, or fermentation products thereof, whose significant function in food is flavoring rather than nutritional. In one aspect, the beverages disclosed herein include one or more added natural flavors. In an alternative aspect, the beverages disclosed herein are free from any added natural flavors other than those already possessed by the ingredients described herein. In another aspect, the beverage further contains a prebiotic.

### Prebiotics

As used herein, “prebiotic” refers to a food ingredient that promotes the growth of beneficial microorganisms (such as, for example, microorganisms consumed as probiotics) in the intestine. In one aspect, the prebiotic can be an inulin or can be maltodextrin, or a combination thereof. In one aspect, the maltodextrin, when included, is a non-GMO digestion-resistant maltodextrin.

In one aspect, the prebiotic is derived from acacia gum or gum arabic, raw dandelion greens, raw leeks, Jerusalem artichokes, raw jicama, underripe bananas, raw chicory root, raw garlic, onions, agave nectar, raw wheat bran, whole wheat flour, yacon, Echinacea, burdock, globe artichoke, wild yam, mugwort, or raw asparagus. In one aspect, the prebiotic is or contains an inulin. In another aspect, the prebiotic can be a “galactooligosaccharide” or “GOS.”

As used herein, “inulin” refers to a naturally occurring polysaccharide produced by a plant such as, for example, chicory. An inulin is a type of dietary fiber and is classified as a “fructan” or a polymer of fructose. A “fructooligosaccharide” or “FOS” or “oligofructose” is a fructan with a short chain length. As an example, the degree of polymerization in inulin can be from 10 to 60, or in some instances, can be up to several thousand fructose units, whereas the degree of polymerization in FOS produced from the chemical degradation of inulin can be from 1 to 14 or from 1 to 7. FOS may improve the flavor or odor of food products; FOS is often used as alternative sweeteners, but FOS, like inulin, can serve as a substrate for probiotic microorganisms in the large intestine.

As used herein, “maltodextrin” is a polysaccharide produced from plant starches such as, for example, corn or wheat starches. In a further aspect, in addition to acting as a prebiotic, when included in the beverages disclosed herein, maltodextrin can alter the thickness or mouthfeel of the beverages. In a still further aspect, when included in the beverages disclosed herein, the maltodextrin can add sweetness to the beverages, or may remain largely flavorless.

In one aspect, the beverages described herein are “symbiotic.” This term refers to products containing both probiotics and prebiotics and specifically to products wherein the prebiotics are chosen selectively to favor the growth of the probiotic microorganisms.

### Properties and Manufacture of the Beverages

In one aspect, the components of the beverages disclosed herein can be admixed in any order using techniques known in the art. “Admixing” or “admixture” refers to a combination of two components together when there is no chemical reaction or physical interaction. The terms “admixing” and “admixture” can also include the chemical interaction or physical interaction between any of the components described herein upon mixing to produce the composition. The components can be admixed alone or in water. As with a beverage, the components are homogeneously dispersed throughout the beverage.

In one aspect, the beverages disclosed herein require refrigeration before and/or after opening. In another aspect, the beverages disclosed herein do not require refrigeration.

In one aspect, the beverages are composed primarily of water, which is referred to herein as “water-based beverage.” In one aspect, the beverage is at least 90% by volume, at least 95% by volume, or at least 98% by volume of the beverage. In another aspect, the beverages described herein have from 5 calories to 20 calories per 8 ounces of beverage

### Benefits of Consuming the Beverages

The beverages described provide numerous health benefits. In one aspect, the beverages described herein can treat or prevent one or more symptoms caused by a bacterial or viral infection (e.g., influenza, coronavirus). In one aspect, the beverages described herein can be used to treat or prevent a number of respiratory disorders including cystic fibrosis, bronchiectasis, rhinitis (both allergic and perennial), sinusitis, emphysema and chronic bronchitis (COPD), acute lung injury/adult respiratory distress syndrome, interstitial lung fibrosis, SARS, asthma, and respiratory syncytial virus. In other aspects, the beverages described herein can prevent and treat infection by common respiratory pathogens (Stretococcus pneumoniae, Hemophilus influenzae, Staphylococcus, Mycoplasma pneumoniae, Chlamydial pneumonia, Gram negative enteric infections) in immune suppressed hosts such as subjects who are HIV positive or who have hematopoietic malignancies. The beverages described herein can treat or alleviate a number of different symptoms including, but not limited to, sinus congestion, shortness of breath, fever, and respiratory congestion.

In one aspect, the beverages described herein are useful for fighting or reducing inflammation. “Inflammation” as used herein is part of the body's response to harmful stimuli. In the instance of injury or tissue damage, inflammation is a protective response localized to a specific area (such as, for example, redness and swelling around a burn). In one aspect, inflammation can caused by a bacterial or viral infection. For example, the beverages can treat or reduce inflammation in the sinuses, throat, and lungs.

In another aspect, consumption of the beverages can correct or improve cases of dysbiosis. As used herein, “dysbiosis” refers to an imbalance of the microbial population on or inside the body, such as the intestinal tract. In some aspects, when a subject is in a state of dysbiosis, pathogenic bacteria normally present in low amounts flourish. This can lead to improper digestion, malabsorption of food and/or specific nutrients, and other illnesses and conditions due to competition between pathogenic bacteria and useful bacteria such as, for example, those that produce certain vitamins (e.g., vitamin K) from dietary precursors. In another aspect, accumulation of waste products from pathogenic bacteria is another harmful effect of dysbiosis. Prolonged dysbiosis can result from inappropriate diet, antibiotic exposure, alcohol abuse, and the like. Dysbiosis in the intestinal tract is best known, but a state of dysbiosis can be found among skin flora (leading to, for example, staph infections such as boils, impetigo, cellulitis, and staphylococcal scalded skin syndrome) and in the vagina (leading to, for examples, vaginal yeast infections). In one aspect, provided herein is a method for treating or preventing dysbiosis in a subject, wherein the method includes the step of consuming the any beverages disclosed herein.

In certain circumstances, dysbiosis can be caused by a bacterial or viral infection of the respiratory system. In other circumstances, dysbiosis can render the subject more susceptible to respiratory infection (bacterial or viral). Thus, the beverages described herein provide a good defense in the prevention of respiratory and gastrointestinal disorders caused by bacterial or viral infection.

The beverages described herein are also useful in enhancing and maintaining the immune system in a subject. Several components present in the beverages described herein can improve immune response in a subject. When consumed collectively, the components present in the beverage provide enhanced immune response.

### Exemplary Aspects

Aspect 1: A beverage comprising (a) water, (b) eucalyptus, (c) ginger or ginger extract, (d) peppermint, (e) elderberry, (f) one or more vitamins, (g) one or more minerals, and (h) a probiotic.
Aspect 2: The beverage of aspect 1, wherein eucalyptus is present in the amount of from about 0.5 mg to about 20 mg per 8 ounce serving of the beverage.
Aspect 3: The beverage of aspect 1 or 2, wherein ginger or ginger extract is present in the amount of from about 0.1 g to about 2 g per 8 ounce serving of the beverage.
Aspect 4: The beverage in any one of aspects 1 to 3, wherein peppermint is present in the amount of from about 5 mg to about 100 mg per 8 ounce serving of the beverage.
Aspect 5: The beverage in any one of aspects 1 to 4, wherein elderberry is present in the amount of from about 1 g to about 20 g per 8 ounce serving of the beverage.
Aspect 6: The beverage in any one of aspects 1 to 5, wherein the vitamin comprises vitamin A, B₁, B2, B₃, B₅, B₆, B₇, B₉, B₁₂, C, D, E, or any combination thereof.
Aspect 7: The beverage in any one of aspects 1 to 6, wherein the vitamin comprises vitamin B₆, C, and D.
Aspect 8: The beverage in any one of aspects 1 to 7, wherein the vitamin comprises B₆ in the amount of from about 0.1 mg to about 2 mg per 8 ounce serving of the beverage, and vitamin D in the amount of from about 1 μg to about 20 μg per 8 ounce serving of the beverage.
Aspect 9: The beverage in any one of aspects 1 to 8, wherein the mineral comprises a pharmaceutically-acceptable salt of calcium, chromium, copper, iron, magnesium, manganese, molybdenum, nickel, potassium, selenium, sodium, vanadium, cobalt, zinc, or any combination thereof.
Aspect 10: The beverage in any one of aspects 1 to 9, wherein the mineral comprises a pharmaceutically-acceptable salt of zinc.
Aspect 11: The beverage in any one of aspects 1 to 10, wherein mineral is present in the amount of from about 1 mg to about 20 mg per 8 ounce serving of the beverage.
Aspect 12: The beverage in any one of aspects 1 to 11, wherein the probiotic comprises Lactobacillus rhamnosus GG (LGG), Lactobacillus casei Shirota (LcS), or a combination thereof.
Aspect 13: The beverage in any one of aspects 1 to 12, wherein the total amount of probiotic is from about 1×10⁹ CFU to about 1×10¹⁰ CFU per 8 ounces of beverage.
Aspect 14: The beverage in any one of aspects 1 to 13, wherein the beverage further comprises a prebiotic.
Aspect 15: The beverage of aspect 14, wherein the prebiotic comprises an inulin or maltodextrin.
Aspect 16: The beverage in any one of aspects 1 to 15, wherein the beverage further comprises a fruit juice concentrate, cane sugar, a stevia extract, polydextrose, a vegetable juice, salt, a natural flavor, a sweetener, preservative, or any combination thereof.
Aspect 17: The beverage in any one of aspects 1 to 15, wherein the beverage does not include a preservative.
Aspect 18: The beverage in any one of aspects 1 to 17, wherein the beverage has from 5 calories to 20 calories per 8 ounces of beverage.
Aspect 19: The beverage in any one of aspects 1 to 18, wherein the beverage comprises:
(a) water;
(b) eucalyptus in the amount of from about 0.5 mg to about 20 mg per 8 ounce serving of the beverage, about 3 mg to about 10 mg, about 4 mg to 6 mg, or about 5 mg;
(c) ginger or ginger extract in the amount of from about 0.1 g to about 2 g per 8 ounce serving of the beverage, about 0.1 mg to about 5 mg, about 0.1 mg to about 1 mg, or about 0.5 mg;
(d) peppermint in the amount of from about 5 mg to about 100 mg per 8 ounce serving of the beverage, about 10 mg to about 80 mg, about 40 mg to about 60 mg, or about 50 mg;
(e) elderberry in the amount of from about 1 mg to about 20 mg per 8 ounce serving of the beverage, about 5 mg to about 15 mg, about 8 mg to about 12 mg, or about 10 mg;
(f) vitamin B₆ in the amount of from about 0.1 mg to about 2 mg per 8 ounce serving of the beverage, about 0.1 mg to about 1 mg, about 0.4 mg to about 0.6 mg, or about 0.5 mg;
(g) vitamin D in the amount of from about 1 μg to about 20 μg per 8 ounce serving of the beverage, about 1 μg to about 10 μg, about 5 μg to about 7 μg, or about 6 μg.
(h) a pharmaceutically-acceptable salt of zinc present in the amount of from about 1 mg to about 20 mg per 8 ounce serving of the beverage, about 1 mg to about 10 mg, about 4 mg to about 6 mg, or about 5 mg; and
(i) a probiotic comprising Lactobacillus rhamnosus GG (LGG) and Lactobacillus casei Shirota (LcS) in the total amount of from about 3×10⁹ CFU to about 9×10⁹ CFU per 8 ounces of beverage, or about 5×10⁹ CFU to about 8×10⁹ CFU.
   Aspect 20: The beverage in any one of aspects 1 to 18, wherein the beverage comprises:
(a) water;
(b) eucalyptus in the amount of from about 0.5 mg to about 20 mg per 8 ounce serving of the beverage, about 3 mg to about 10 mg, about 4 mg to 6 mg, or about 5 mg;
(c) ginger or ginger extract in the amount of from about 0.1 g to about 2 g per 8 ounce serving of the beverage, about 0.1 mg to about 5 mg, about 0.1 mg to about 1 mg, or about 0.5 mg;
(d) peppermint in the amount of from about 5 mg to about 100 mg per 8 ounce serving of the beverage, about 10 mg to about 80 mg, about 40 mg to about 60 mg, or about 50 mg;
(e) elderberry in the amount of from about 1 g to about 20 g per 8 ounce serving of the beverage, about 5 g to about 15 g, about 8 g to about 12 g, or about 10 g;
(f) vitamin B₆ in the amount of from about 0.1 mg to about 2 mg per 8 ounce serving of the beverage, about 0.1 mg to about 1 mg, about 0.4 mg to about 0.6 mg, or about 0.5 mg;
(g) vitamin D in the amount of from about 1 μg to about 20 μg per 8 ounce serving of the beverage, about 1 μg to about 10 μg, about 5 μg to about 7 μg, or about 6 μg.
(h) a pharmaceutically-acceptable salt of zinc present in the amount of from about 1 mg to about 20 mg per 8 ounce serving of the beverage, about 1 mg to about 10 mg, about 4 mg to about 6 mg, or about 5 mg; and
(i) a probiotic comprising Lactobacillus rhamnosus GG (LGG) and Lactobacillus casei Shirota (LcS) in the total amount of from about 3×10⁹ CFU to about 9×10⁹ CFU per 8 ounces of beverage, or about 5×10⁹ CFU to about 8×10⁹ CFU.
   Aspect 21: A method for treating or preventing one or more symptoms caused by a bacterial or viral infection in a subject comprising consuming the beverage in any one of aspects 1 to 20.
   Aspect 22: The method of aspect 21, wherein the infection comprises a sinus infection, a respiratory infection, or a gastrointestinal infection.
   Aspect 23: The method of aspects 21 or 22, wherein the symptom comprises sinus congestion, shortness of breath, fever, or respiratory congestion.
   Aspect 24: The method in any one of aspects 21 to 23, wherein the subject has respiratory disorder.
   Aspect 25: A method for enhancing the immune system in a subject comprising consuming the beverage in any one of aspects 1 to 20.

Throughout this application, various publications are referenced. The disclosures of these publications in their entireties are hereby incorporated by reference into this application in order to more fully describe the compounds, compositions, and methods described herein.

Various modifications and variations can be made to the compounds, compositions, and methods described herein. Other aspects of the compounds, compositions, and methods described herein will be apparent from consideration of the specification and practice of the compounds, compositions, and methods disclosed herein. It is intended that the specification and examples be considered as exemplary.

What is claimed:

1. A beverage comprising (a) water, (b) eucalyptus, (c) ginger or ginger extract, (d) peppermint, (e) elderberry, (f) one or more vitamins, (g) one or more minerals, and (h) a probiotic.

2. The beverage of claim 1, wherein eucalyptus is present in the amount of from about 0.5 mg to about 20 mg per 8 ounce serving of the beverage.

3. The beverage of claim 1, wherein ginger or ginger extract is present in the amount of from about 0.1 g to about 2 g per 8 ounce serving of the beverage.

4. The beverage of claim 1, wherein peppermint is present in the amount of from about 5 mg to about 100 mg per 8 ounce serving of the beverage.

5. The beverage of claim 1, wherein elderberry is present in the amount of from about 1 g to about 20 g per 8 ounce serving of the beverage.

6. The beverage of claim 1, wherein the vitamin comprises vitamin A, B₁, B₂, B₃, B₅, B₆, B₇, B₉, B₁₂, C, D, E, or any combination thereof.

7. The beverage of claim 1, wherein the vitamin comprises vitamin B₆, C, and D.

8. The beverage of claim 1, wherein the vitamin comprises B₆ in the amount of from about 0.1 mg to about 2 mg per 8 ounce serving of the beverage, and vitamin D in the amount of from about 1 μg to about 20 μg per 8 ounce serving of the beverage.

9. The beverage of claim 1, wherein the mineral comprises a pharmaceutically-acceptable salt of calcium, chromium, copper, iron, magnesium, manganese, molybdenum, nickel, potassium, selenium, sodium, vanadium, cobalt, zinc, or any combination thereof.

10. The beverage of claim 1, wherein the mineral comprises a pharmaceutically-acceptable salt of zinc.

11. The beverage of claim 1, wherein mineral is present in the amount of from about 1 mg to about 20 mg per 8 ounce serving of the beverage.

12. The beverage of claim 1, wherein the probiotic comprises Lactobacillus rhamnosus GG (LGG), Lactobacillus casei Shirota (LcS), or a combination thereof.

13. The beverage of claim 1, wherein the total amount of probiotic is from about 1×10⁹ CFU to about 1×10¹⁰ CFU per 8 ounces of beverage.

14. The beverage of claim 1, wherein the beverage further comprises a prebiotic.

15. The beverage of claim 14, wherein the prebiotic comprises an inulin or maltodextrin.

16. The beverage of claim 1, wherein the beverage further comprises a fruit juice concentrate, cane sugar, a stevia extract, polydextrose, a vegetable juice, salt, a natural flavor, a sweetener, preservative, or any combination thereof.

17. The beverage of claim 1, wherein the beverage does not include a preservative.

18. The beverage of claim 1, wherein the beverage comprises:
(j) water;
(k) eucalyptus in the amount of from about 0.5 mg to about 20 mg per 8 ounce serving of the beverage, about 3 mg to about 10 mg, about 4 mg to 6 mg, or about 5 mg;
(l) ginger or ginger extract in the amount of from about 0.1 g to about 2 g per 8 ounce serving of the beverage, about 0.1 mg to about 5 mg, about 0.1 mg to about 1 mg, or about 0.5 mg;
(m) peppermint in the amount of from about 5 mg to about 100 mg per 8 ounce serving of the beverage, about 10 mg to about 80 mg, about 40 mg to about 60 mg, or about 50 mg;
(n) elderberry in the amount of from about 1 mg to about 20 mg per 8 ounce serving of the beverage, about 5 mg to about 15 mg, about 8 mg to about 12 mg, or about 10 mg;
(o) vitamin B₆ in the amount of from about 0.1 mg to about 2 mg per 8 ounce serving of the beverage, about 0.1 mg to about 1 mg, about 0.4 mg to about 0.6 mg, or about 0.5 mg;
(p) vitamin D in the amount of from about 1 μg to about 20 μg per 8 ounce serving of the beverage, about 1 μg to about 10 μg, about 5 μg to about 7 μg, or about 6 μg.
(q) a pharmaceutically-acceptable salt of zinc present in the amount of from about 1 mg to about 20 mg per 8 ounce serving of the beverage, about 1 mg to about 10 mg, about 4 mg to about 6 mg, or about 5 mg; and
(r) a probiotic comprising Lactobacillus rhamnosus GG (LGG) and Lactobacillus casei Shirota (LcS) in the total amount of from about 3×10⁹ CFU to about 9×10⁹ CFU per 8 ounces of beverage, or about 5×10⁹ CFU to about 8×10⁹ CFU.

19. A method for treating or preventing one or more symptoms caused by a bacterial or viral infection in a subject comprising consuming the beverage of claim 1.

20. A method for enhancing the immune system in a subject comprising consuming the beverage of claim 1.
